(19) United States
(12) Patent Application Publication
(10) Pub. No.: US 2025/0354977 A1
(43) Pub. Date: Nov. 20, 2025

(54) CLOSED GAS EXCHANGE TRANSIENT BUFFERING SYSTEMS AND METHODS

(71) Applicant: LI-COR, Inc., Lincoln, NE (US)

(72) Inventors: Johnathan McCoy, Lincoln, NE (US); Liukang Xu, Lincoln, NE (US); Jason Hupp, Lincoln, NE (US)

(21) Appl. No.: 18/668,570

(22) Filed: May 20, 2024

(51) Int. Cl. G01N 33/497

(52) U.S. Cl. CPC G01N 33/4977

(57) ABSTRACT

Gas exchange analysis methods and systems utilize a buffering component including a material configured to buffer water vapor in a flow of a gas, whereby fluctuations in the water vapor content in the flow of the gas are minimized or reduced in magnitude for components in the flow path. Components in the flow path may include: a gas analyzer configured to receive a flow of a gas from a first gas flow line coupled to an exit of a sample chamber and configured to measure a first concentration of an analyte of interest in the flow of the gas received in the first gas flow line from the sample chamber; the sample chamber, which is configured to hold a sample capable of adding or removing water from the gas and configured to receive the gas exiting the gas analyzer after measurement by the gas analyzer; and the buffering component positioned in the first gas flow line between the gas analyzer and the sample chamber.

### BACKGROUND

Gas exchange measurement systems, such as systems for measuring gas flux from the soil or a plant canopy, can be categorized as open or closed systems. For open systems, a leaf or sample may be enclosed in a sample chamber, and an air stream is passed continuously through the chamber. CO₂, or other gases, and H₂O concentrations of chamber influent and effluent are measured, and the difference between influent and effluent concentration is calculated. (Throughout this document the term “concentration” refers to mole fraction of a gas in natural or synthetic moist air, or mole fraction in natural or synthetic dry air (“dry mole fraction”) where such is specified.) This difference may be used, along with the mass flow rate, to calculate metrics of interest such as carbon efflux (CO₂) and evapotranspiration (H₂O) rates or fluxes of other gases. For closed systems, an analysis chamber enclosing a leaf or plant or other material may be supplied with air and the system then closed (not supplied with fresh air). The concentrations of CO₂, or other gases, and H₂O are continuously monitored within the chamber as air circulates throughout the enclosed system. The rate of change of these concentrations, along with the system volume (chamber volume and recirculation volume), may be used to calculate carbon efflux (CO₂) rate, and fluxes of other gas constituent or analytes of interest such as methane or other trace gases.

During the time of measurement in a closed system, the H₂O concentration can also increase in the presence of a sample, resulting in interference of the measurement of the gas or analyte of interest and errors in the calculated fluxes.

Thus, there is a need for improved gas exchange analysis systems and methods for that may eliminate or reduce the effects of changes in water vapor or other interfering analyte during closed system gas measurements.

### SUMMARY

The present disclosure provides systems and methods for reducing or minimizing the impact of water vapor or other analyte on measurements in a closed gas exchange measurement system. More generally, embodiments dampen or smooth-out changes in water vapor.

Various embodiments herein minimize the increase, or decrease, in H₂O or other interfering analyte during the measurement period without interaction with other gases or analytes of interest. Consequently, the embodiments herein advantageously reduce the interference of H₂O or other interfering analyte on measurements of an analyte of interest during the measurement while consuming no power and not needing any user interaction or user maintenance. The present embodiments are useful for long-term remotely deployed situations.

According to an embodiment, a closed gas exchange analysis system is provided that includes a gas analyzer configured to receive a flow of a gas from a first gas flow line coupled to an exit of a sample chamber and configured to measure a first concentration of an analyte of interest in the flow of the gas received in the first gas flow line from the sample chamber, and the sample chamber, wherein the sample chamber is configured to hold a sample capable of adding or removing water from the gas and configured to receive the gas exiting the gas analyzer after measurement by the gas analyzer. The system also includes a component in the first gas flow line between the gas analyzer and the sample chamber, the component including an amount of a material configured to buffer water vapor content in the flow of the gas, whereby changes in water vapor content in the flow of the gas measured by the gas analyzer are reduced or minimized in magnitude.

In certain aspects, the material absorbs water in the presence of a positive water concentration gradient and desorbs water in the presence of a negative water concentration gradient to thereby control a rate of change in water vapor content propagated in the flow of the gas to the gas analyzer from the sample chamber.

In certain aspects, the material absorbs or desorbs water in the presence of a water concentration gradient. In certain aspects, the material includes a Nafion™ structure. Nafion is a sulfonated tetrafluoroethylene based fluoropolymer-copolymer developed by DuPont. In certain aspects, the structure is selected from the group consisting of one or more beads, a tube and a flat membrane. In certain aspects, the material includes one or a plurality of Nafion beads. In certain aspects, the buffering component includes a mechanism to adjust an amount of the material in the gas flow line, for example, the mechanism may be configured for dynamic adjustment of an amount of the material in the gas flow line. In certain aspects, the measurement airstream encompasses all surfaces of the Nafion structure.

In certain aspects, the analyte of interest includes CO₂ or CH₄ or N₂O or their isotopolouges.

In certain aspects, the closed gas exchange measurement system includes a second gas analyzer configured to receive the flow of the gas from the first gas flow line coupled to the exit of a sample chamber and configured to measure a first concentration of a second analyte of interest in the flow of the gas received in the first gas flow line from the sample chamber. The (first) and second gas analyzers may be arranged in parallel and/or in series with each other to analyze gas in the flow line exiting the sample chamber and buffer component.

In certain aspects, the sample includes a photosynthesis and/or transpiration capable material.

In certain aspects, each gas analyzer may include one of a capacitive sensor, a resistive sensor, a thermal-conductivity-based sensor, an optical absorption gas analyzer, or a laser-based gas analyzer.

According to an embodiment, a method of measuring a gas flux in a closed gas exchange measurement system is provided. The method includes providing an internal volume of a sample chamber with ambient air from an environment external to the sample chamber, the sample chamber containing a sample capable of adding or removing water from the gas, closing the sample chamber to the environment, and thereafter continuously measuring, for a period of time while the sample chamber is closed to the environment, a concentration of an analyte of interest in the flow of the gas received in a first gas flow line from the sample chamber, wherein the first gas flow line includes a component including an amount of a material configured to buffer water vapor content in the flow of the gas, whereby changes in water vapor content in the flow of the gas measured by the gas analyzer are minimized or reduced in magnitude.

In certain aspects, the material absorbs water in the presence of a positive water concentration gradient and desorbs water in the presence of a negative water concentration gradient to thereby control a rate of changes in water vapor content propagated in the flow of the gas to the gas analyzer from the sample chamber.

In certain aspects, the material includes a Nafion material. In certain aspects, the Nafion material has a structure selected from the group consisting of one or more beads, a tube and a membrane.

In certain aspects, the material includes one or a plurality of Nafion beads.

In certain aspects, the analyte of interest includes CO₂ or CH₄ or N₂O or their isotopolouges.

In certain aspects, the gas measured by the gas analyzer is continuously reintroduced back into the sample chamber during the continuously measuring, e.g., using a pump or other mechanism in a flow line.

In certain aspects, the gas analyzer includes one of a capacitive sensor, a resistive sensor, a thermal-conductivity-based sensor, an optical absorption gas analyzer, or a laser-based gas analyzer.

According to another embodiment, a closed gas exchange analysis system is provided that includes a gas analyzer configured to receive a flow of a gas from a first gas flow line coupled to an exit of a sample chamber and configured to measure a first concentration of a first analyte in the flow of the gas received in the first gas flow line from the sample chamber, and the sample chamber, wherein the sample chamber is configured to hold a sample capable of adding or removing a second analyte from the gas and configured to receive the gas exiting the gas analyzer after measurement by the gas analyzer. The system also includes a component in the first gas flow line between the gas analyzer and the sample chamber, the component including an amount of a material configured to buffer the second analyte in the flow of the gas, whereby changes in concentration of the second analyte in the flow of the gas measured by the gas analyzer are minimized or reduced in magnitude.

In certain aspects, the material absorbs the second analyte in the presence of a positive second analyte concentration gradient and desorbs the second analyte in the presence of a negative second analyte concentration gradient to thereby control a rate of change in the second analyte concentration propagated in the flow of the gas to the gas analyzer from the sample chamber.

In certain aspects, the first analyte includes CO₂ or CH₄ or N₂O or their isotopolouges, and wherein the second analyte includes H₂O.

As used herein, adding water content or removing water content from an air stream may include outgassing, desorbing, a chemical reaction, a metabolic reaction, or other mechanisms for generating or removing water content.

In certain aspects, a sample may include any material, substance or organism that exchanges, generates or consumes water or other analyte. In certain aspects, the sample may include a water saturable or aqueous sample, which may include a photosynthesis capable material, substance or organism, such as a leaf or algae, or may include a respiratory material, substance or organism, e.g., a material that respires, or may include a metabolically active material, substance or organism.

In a further embodiment, a non-transitory computer readable medium is provided that stores instructions, which when executed by one or more processors, cause a system including the one or more processors to implement a method of measuring a concentration of a gas, and computing relevant variables such as a gas flux, using a gas exchange measurement system as described herein.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE SEVERAL VIEWS OF THE DRAWINGS

The detailed description is described with reference to the accompanying figures. The use of the same reference numbers in different instances in the description and the figures may indicate similar or identical items.

FIG. 1 illustrates a simplified flow and a physical arrangement of a closed gas exchange measurement system including an analyte buffering component according to an embodiment.

FIG. 2 shows an example of a buffer component embodiment including a single continuous flow path having four legs, each containing multiple Nafion beads.

FIG. 3 and FIG. 4 demonstrate the effectiveness of Nafion in slowing the rate of incoming water vapor content for lengths of tubing including 75 NR50 Nafion beads and 90 NR50 Nafion beads, respectively.

### DETAILED DESCRIPTION

The present disclosure provides systems and method for automatically reducing or minimizing the impact of water vapor changes may have on measurements in a closed-path (“closed”) gas exchange measurement system. For example, embodiments herein provide a buffer component in a closed-transient system where one (or multiple) analyzers may be used, which buffer component advantageously keeps water vapor concentration levels constant, or nearly constant, to eliminate measurement errors in a gas of interest due to cross-sensitivity in the water corrections, both spectroscopic and in accounting for volumetric dilution due to water vapor.

In an example closed gas exchange measurement system, air is provided to a system volume, which may include a sample chamber, e.g., cuvette, containing material that adds or removes water vapor to the system volume, a gas analyzer and a flow path (e.g., tubing) connecting the sample chamber and gas analyzer. Measurement errors in this system may result from spectroscopic or other fundamental interactions between water vapor and the analyte of interest. These interactions may be well accounted for under steady-state water vapor concentrations, but less so under transient conditions. Errors due to the water vapor transient can propagate into the flux, leading to errors in it and complicating its interpretation. A constant concentration in water vapor concentration during the measurement is most often desired.

Embodiments advantageously alleviate this concern by reducing or slowing, in a controllable and/or user-selectable fashion, changes in water vapor concentration in the system volume that may be attributable to a sample material. Certain embodiments use a specific material, Nafion™, to buffer water vapor content in the flow path of the gas exchange system. Nafion is a material that has a capacity to store water vapor. Nafion selectively exchanges water vapor in the presence of a concentration gradient following first order kinetics, and equilibrium is reached within milliseconds.

According to various embodiments, placing a certain quantity of Nafion in the flow-path, and ensuring proper interaction between the sample exit flow and the Nafion, allows the water vapor in the flow path to be influenced by the water content held by the Nafion material, which is in turn influenced by previous water vapor concentration. The volume and surface area of the Nafion exposed to the flow path may be optimized to reduce the amplitude of transients which are propagated. Increasing water vapor concentration in the flow path will be absorbed by the Nafion, resulting in a slower rate of increase in the water concentration interacting with components in the system volume, e.g., sample chamber and gas analyzer(s). Conversely, a decreasing water vapor concentration will desorb water from the Nafion, resulting in a slower rate of decrease in the water concentration interacting with downstream components. In this manner, the rate of water concentration changes in the flow path are slowed by the Nafion.

It will be appreciated that any other material that selectively buffers water vapor content may be used, for example, any water vapor selective element or material that allows water or water vapor to pass in a uni-directional or bi-directional manner dependent on the water concentration gradient at the surface of interaction.

It will also be appreciated that other analytes or gases of interest may be buffered using a material that selectively buffers the particular analyte or gas, e.g., a material which mimics the first-order kinetics of Nafion/water for that particular gas or analyte.

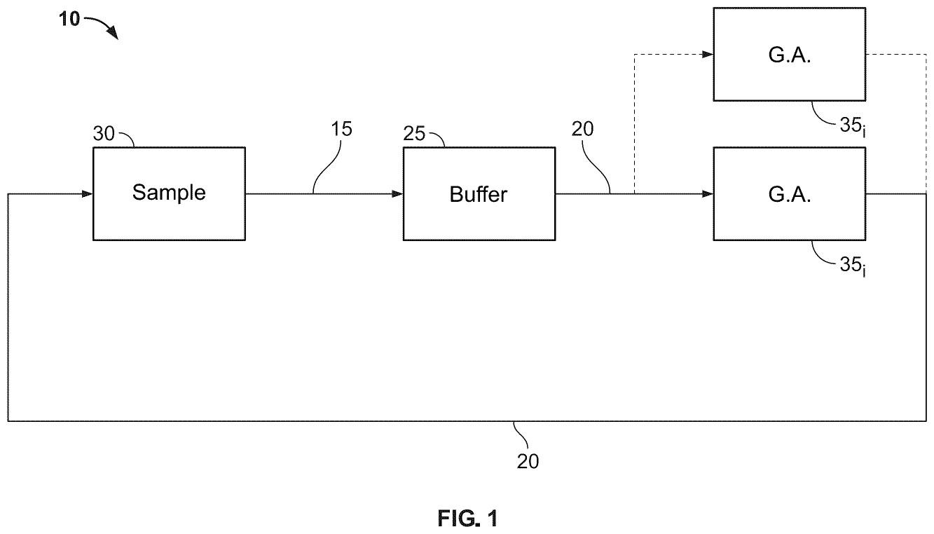

FIG. 1 shows a simplified closed gas exchange measurement system 10 and gas flow diagram according to an embodiment. A buffering component 25 receives a flow (e.g., airstream) of gas exiting from the sample chamber 30 in flow line 15 and provides a gas flow to downstream components via gas flow line 20. In the embodiment shown in FIG. 1, the system components are arranged in series and include a sample chamber 30, a buffer component 25 and one or more gas analyzers 35i (i=1 to N, number of gas analyzers) arranged in parallel and/or in series to flow path. Flow path 20 recirculates the gas flow back to the sample chamber 30, such that the system volume includes sample chamber 30, flow lines 14 and 20 and gas analyzer(s) 35i. A pump or other mechanism may be provided in a flow line to facilitate recirculation of the gas back through sample chamber 30.

Buffering component 25 includes an analyte selective component, element or material and effectively acts as a conditioned source of gas with any changes in intensity of the analyte content in the incoming flow of gas smoothed out or slowed for the downstream components. For example, where the interfering analyte is H2O, the buffering component 25 includes a water selective component, element or material and effectively acts as a conditioned source of gas with any changes in intensity of the water vapor content in the incoming flow of gas smoothed out or slowed for the downstream components such as gas analyzers 35i. For example, a water vapor selective element may include a Nafion structure configured to interact with the airstream received from sample chamber 30. The Nafion structure may be configured as a one or a plurality of Nafion beads, or one or more Nafion sheets or as a tubular Nafion structure, or it may take on any other geometric configuration.

In operation, air or other gas is introduced into the sample chamber 30 (which typically will include a sample for analysis) and/or system volume. The gas in the sample volume interacts with any sample material present in sample chamber 30. Such interaction may result in analyte, e.g., water vapor, being added to, or removed from, the gas flow. The gas analyzers 35i measure the gas flow exiting the sample chamber 30.

Each gas analyzer 35 may include a water vapor sensing mechanism and/or a mechanism for sensing a different analyte of interest. For example, each gas analyzer may include a humidity sensor or a different type of water vapor sensor. Examples of water vapor sensors include capacitive humidity sensors, resistive humidity sensors, thermal conductivity-based humidity sensors, hygrometers, optical humidity sensors, such as laser-based sensors, or other sensors as would be apparent to one skilled in the art.

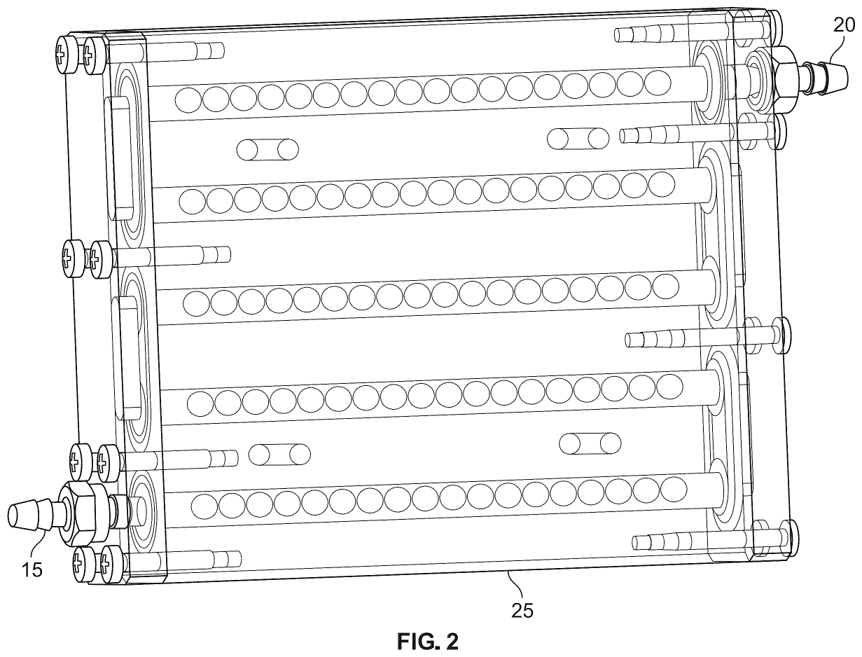

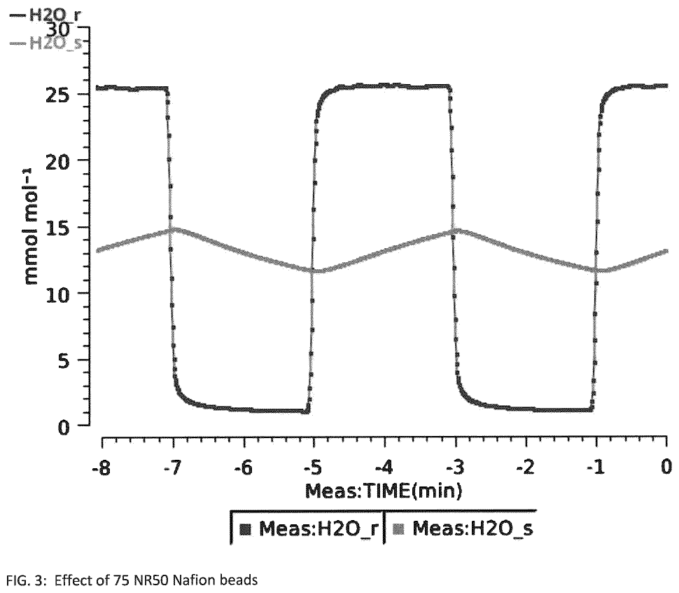

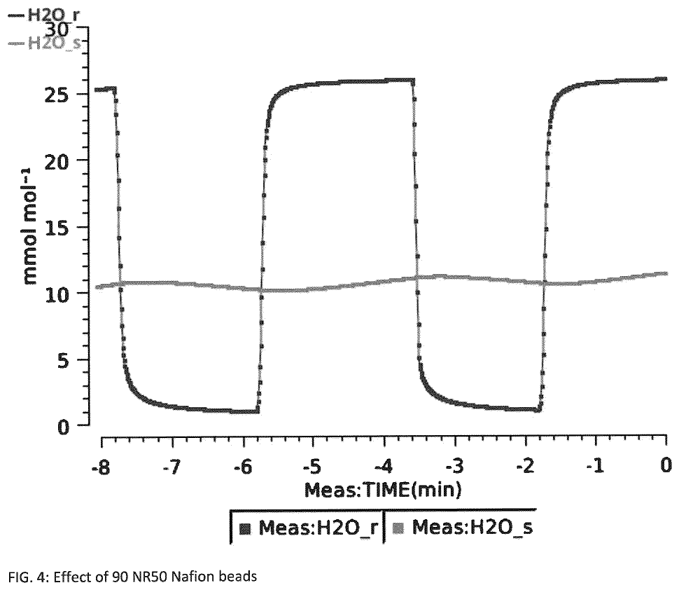

The effectiveness of using Nafion to buffer water vapor in an airstream has been demonstrated using an experimental arrangement including short lengths of tubing containing a number of spherical Nafion beads. The beads were installed in-line in the flow-path between a gas exchange chamber and analyzer, e.g., a non-dispersive infra-red (NDIR) gas analyzer, of a LI-6800 portable photosynthesis instrument to simulate “worst case” water vapor transients (a step change) in a closed system. The buffer was made using a length of plastic tubing with 3 mm diameter beads placed in series as shown in FIG. 2, which shows an example of a buffer component embodiment including a single continuous flow path having four legs, each containing multiple Nafion beads. Changes to the in-coming water vapor content (H2O_r) were dynamically controlled. FIG. 3 and FIG. 4 demonstrate the effectiveness of Nafion in slowing the rate of outgoing water vapor content (H2O_s) for lengths of tubing including 75 NR50 Nafion beads and 90 NR50 Nafion beads, respectively.

In certain embodiments, a control system (not shown), e.g., including one or more processors and associated memory, may be provided to control various system components, e.g., to close the system volume (e.g., using controllable valves) and to control the gas analyzers to initiate real-time water gas concentration measurements.

In certain embodiments, the control system or other intelligence module, which may include a processing component or components such as one or more processors and associated memory and/or storage, is configured to control, and to receive and process data from, the gas analyzers to implement the methods disclosed herein, e.g., real-time concentration measurements of water vapor concentration and/or other analyte concentration or properties of interest.

Each processor or processing component is configured to implement functionality and/or process instructions for execution, for example, instructions stored in memory or instructions stored on storage devices, and may be implemented as an ASIC including an integrated instruction set. A memory, which may be a non-transient computer-readable storage medium, is configured to store information during operation. In some embodiments, a memory includes a temporary memory or area for information not to be maintained when the processing component is turned OFF. Examples of such temporary memory include volatile memories such as random access memories (RAM), dynamic random access memories (DRAM), and static random access memories (SRAM). The memory maintains program instructions for execution by the processing component.

Storage devices also include one or more non-transient computer-readable storage media. Storage devices are generally configured to store larger amounts of information than the memory. Storage devices may further be configured for long-term storage of information. In some examples, storage devices include non-volatile storage elements. Non-limiting examples of non-volatile storage elements include magnetic hard disks, optical discs, floppy discs, flash memories, or forms of electrically programmable memories (EPROM) or electrically erasable and programmable (EEPROM) memories.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosed subject matter (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or example language (e.g., “such as”) provided herein, is intended merely to better illuminate the disclosed subject matter and does not pose a limitation on the scope of the invention unless otherwise claimed. No language in the specification should be construed as indicating any non-claimed element as essential to the practice of the invention.

Certain embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A closed gas exchange analysis system, the system comprising:
a gas analyzer configured to receive a flow of a gas from a first gas flow line coupled to an exit of a sample chamber and configured to measure a first concentration of an analyte of interest in the flow of the gas received in the first gas flow line from the sample chamber;
the sample chamber, wherein the sample chamber is configured to hold a sample capable of adding or removing water from the gas and configured to receive the gas exiting the gas analyzer after measurement by the gas analyzer; and
a component in the first gas flow line between the gas analyzer and the sample chamber, the component including an amount of a material configured to buffer water vapor content in the flow of the gas, whereby changes in water vapor content in the flow of the gas measured by the gas analyzer are minimized or reduced in magnitude.

2. The system of claim 1, wherein the material absorbs water in the presence of a positive water concentration gradient and desorbs water in the presence of a negative water concentration gradient to thereby control a rate of change in water vapor content propagated in the flow of the gas to the gas analyzer from the sample chamber.

3. The system of claim 1, wherein the material includes a Nafion structure.

4. The system of claim 3, wherein the Nafion structure is selected from the group consisting of one or more beads, a tube and a flat membrane.

5. The system of claim 2, wherein the material includes one or a plurality of Nafion beads arranged in series and/or in parallel.

6. The system of claim 1, wherein the analyte of interest includes CO₂ or CH₄ or N₂O or their isotopolouges.

7. The system of claim 1, wherein the gas exchange measurement system includes a second gas analyzer configured to receive the flow of the gas from the first gas flow line coupled to the exit of a sample chamber and configured to measure a first concentration of a second analyte of interest in the flow of the gas received in the first gas flow line from the sample chamber.

8. The system of claim 1, wherein the sample includes a biologically active material capable of respiration, photosynthesis and/or evapotranspiration.

9. The system of claim 1, wherein the gas analyzer includes one of a capacitive sensor, a resistive sensor, a thermal-conductivity-based sensor, an optical absorption gas analyzer, or a laser-based gas analyzer.

10. A method of measuring a gas flux in a closed gas exchange measurement system, the method comprising:
providing an internal volume of a sample chamber with ambient air from an environment external to the sample chamber, the sample chamber containing a sample capable of adding or removing water from the gas;
closing the sample chamber to the environment; and thereafter
continuously measuring, for a period of time while the sample chamber is closed to the environment, a concentration of an analyte of interest in the flow of the gas received in a first gas flow line from the sample chamber, wherein the first gas flow line includes a component including an amount of a material configured to buffer water vapor content in the flow of the gas, whereby changes in water vapor content in the flow of the gas measured by the gas analyzer are minimized or reduced in magnitude.

11. The method of claim 10, wherein the material absorbs water in the presence of a positive water concentration gradient and desorbs water in the presence of a negative water concentration gradient to thereby control a rate of changes in water vapor content propagated in the flow of the gas to the gas analyzer from the sample chamber

12. The method of claim 10, wherein the material includes a Nafion material.

13. The method of claim 12, wherein the Nafion material has a structure selected from the group consisting of one or more beads, a tube and a membrane.

14. The method of claim 10, wherein the material includes one or a plurality of Nafion beads.

15. The method of claim 10, wherein the analyte of interest includes CO₂ or CH₄ or N₂O or their isotopolouges.

16. The method of claim 10, wherein the gas measured by the gas analyzer is continuously reintroduced back into the sample chamber during the continuously measuring.

17. The method of claim 10, wherein the gas analyzer includes one of a capacitive sensor, a resistive sensor, a thermal-conductivity-based sensor, an optical absorption gas analyzer, or a laser-based gas analyzer.

18. A closed gas exchange analysis system, the system comprising:
a gas analyzer configured to receive a flow of a gas from a first gas flow line coupled to an exit of a sample chamber and configured to measure a first concentration of a first analyte in the flow of the gas received in the first gas flow line from the sample chamber;
the sample chamber, wherein the sample chamber is configured to hold a sample capable of adding or removing a second analyte from the gas and configured to receive the gas exiting the gas analyzer after measurement by the gas analyzer; and
a component in the first gas flow line between the gas analyzer and the sample chamber, the component including an amount of a material configured to buffer the second analyte in the flow of the gas, whereby changes in concentration of the second analyte in the flow of the gas measured by the gas analyzer are minimized or reduced in magnitude.

19. The system of claim 18, wherein the material absorbs the second analyte in the presence of a positive second analyte concentration gradient and desorbs the second analyte in the presence of a negative second analyte concentration gradient to thereby control a rate of change in the second analyte concentration propagated in the flow of the gas to the gas analyzer from the sample chamber.

20. The system of claim 18, wherein the first analyte includes CO₂ or CH₄ or N₂O or their isotopolouges, and wherein the second analyte includes H₂O.
